(19) United States
(12) Patent Application Publication
(10) Pub. No.: US 2012/0208837 A1
(43) Pub. Date: Aug. 16, 2012

(54) SUBSTITUTED AZAINDOLES

(76) Inventor: Roger Tung, Lexington, MA (US)

(21) Appl. No.: 13/230,970

(22) Filed: Sep. 13, 2011

Related U.S. Application Data
(60) Provisional application No. 61/382,149, filed on Sep. 13, 2010.

(51) Int. Cl. A61K 31/437; A61P 35/02; A61P 7/00; C07D 471/04; A61P 35/00

(52) U.S. Cl. 514/300; 546/113

(57) ABSTRACT

This invention relates to novel substituted azaindoles and pharmaceutically acceptable salts thereof. This invention also provides compositions comprising a compound of this invention and the use of such compositions in methods of treating diseases and conditions that are beneficially treated by administering a compound showing selective inhibitory activity of oncogenic B-Raf^{V600E} protein kinase.

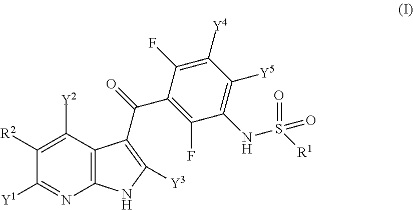

### CROSS-REFERENCE TO RELATED APPLICATIONS

This application claims the benefit of priority to U.S. Provisional Application No. 61/382,149, filed Sep. 13, 2010, the contents of which are incorporated by reference herein in their entirety.

### BACKGROUND OF THE INVENTION

Many current medicines suffer from poor absorption, distribution, metabolism and/or excretion (ADME) properties that prevent their wider use. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to maintain a sufficiently high plasma level of drug. This, however, introduces a number of potential treatment problems such as poor patient compliance with the dosing regimen, side effects that become more acute with higher doses, and increased cost of treatment.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, quinidine has been added to dextromethorphan for the purpose of reducing rapid CYP2D6 metabolism in a treatment of pseudobulbar affect. Quinidine, however, is a CYP2D6 inhibitor that has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism. (See Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of overall metabolism will differ from that of its undeuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

This invention relates to novel deuterated azaindoles and pharmaceutically acceptable salts thereof. This invention also provides compositions comprising a compound of this invention and the use of such compositions in methods of treating diseases and conditions that are beneficially treated by administering a B-Raf^{V600E} protein kinase inhibitor.

PLX4032, also known as N-(3-(5-(4-chlorophenyl)-1H-pyrrolo[2,3-b]pyridine-3-carbonyl)-2,4-difluorophenyl)propane-1-sulfonamide, selectively inhibits oncogenic B-Raf^{V600E} protein kinase (Puzanov, I. et al., J. Clin. Oncol., 2009, 27(15s): suppl; abstr 9021; Flaherty, K. et al., J. Clin. Oncol., 2009, 27(15s): suppl; abstr 9000, Bollag, G. et al., Nature—Letters, 2010, 1-5). Oncogenic mutations in the B-Raf gene have been linked to a variety of cancers. Reportedly, approximately 70% of melanoma cases, 30-70% of thyroid cancer cases, 15-30% of ovarian cancer cases and 5-20% of colorectal cancer cases have been associated with B-Raf-activating mutations (Sala, E. et al., Mol Cancer Res., 2008 May, 6(5): 751-9). A single-base mutation to B-Raf at nucleotide 1799 in codon 600 of exon 15 leading to a valine-to-glutamate substitution (V600E) has been found in more than 85% of melanomas with a B-Raf mutation (Langland, R. et al., EP2036990A.1).

PLX4032 is currently undergoing clinical evaluation for treatment of malignant melanoma and for colorectal cancer (see http://clinicaltrials.gov).

Despite the purported beneficial activities of PLX4032, there is a continuing need for new compounds that have beneficial effects for treatment of malignant melanoma and for colorectal cancer.

### Definitions

The term “treat” means decrease, suppress, attenuate, diminish, arrest, or stabilize the development or progression of a disease (e.g., a disease or disorder delineated herein).

“Disease” means any condition or disorder that damages or interferes with the normal function of a cell, tissue, or organ.

The term “alkyl” refers to a monovalent saturated hydrocarbon group. C₁-C₆ alkyl is an alkyl having from 1 to 6 carbon atoms. C₁-C₄ alkyl is an alkyl having from 1 to 4 carbon atoms. An alkyl may be linear or branched. Examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl.

The term “C₆-C₁₀ aryl” refers to a monovalent aromatic ring system having from 6 to 10 ring carbon atoms. The ring system may be a monocyclic or fused bicyclic ring system. Examples of C₆ aryl. include phenyl. Examples of C₁₀ aryl include naphthyl, including 1-naphthyl and 2-naphthyl.

The term “halo” or “halogen” refers to —Cl, —Br, —F, and —I.

It will be recognized that some variation of natural isotopic abundance occurs in a synthesized compound depending upon the origin of chemical materials used in the synthesis. Thus, a preparation of PLX4032 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes L Z et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species that differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention. A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any salt that is non-toxic upon administration to a recipient at a therapeutically effective dose level, and is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free of another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes specified herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert” and “t-” each refer to tertiary. “US” refers to the United States of America.

The phrase “substituted with deuterium” means that one or more positions in the indicated moiety are substituted with a deuterium atom.

Throughout this specification, a variable may be referred to generally (e.g.,“each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

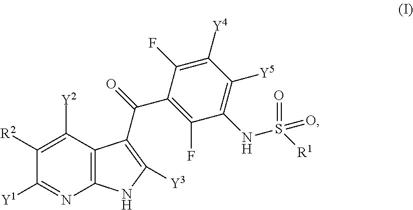

or a pharmaceutically acceptable salt thereof, wherein:

R¹ is C₁-C₄ alkyl optionally substituted with deuterium;

each of Y¹, Y², Y³, Y⁴, and Y⁵ is independently selected from hydrogen and deuterium;

and

R² is C₆-C₁₀ aryl (i) optionally substituted with deuterium and (ii) optionally substituted with halo or with R³; R³ is C₁-C₄ alkyl optionally substituted with deuterium;

with the proviso that if each of Y¹, Y², Y³, Y⁴, and Y⁵ is hydrogen, then

at least one of R¹, R² and R³ comprises deuterium.

In one embodiment, R¹ is isopropyl optionally substituted with deuterium. In one aspect of this embodiment, R¹ is —CH(CH₃)₂, —CD(CH₃)₂, —CH(CD₃)₂, or —CD(CD₃)₂.

In one embodiment, R¹ is n-propyl optionally substituted with deuterium. In one aspect of this embodiment, R¹ is —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃.

In one embodiment, R² is phenyl optionally substituted with halo and optionally substituted with deuterium. In one aspect of this embodiment, R² is phenyl substituted with —Cl at the 4-position and optionally substituted with deuterium. In one aspect of this embodiment, R¹ is n-propyl optionally substituted with deuterium, such as —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃. In a more particular aspect, R² is phenyl substituted with —Cl at the 4-position and optionally substituted with deuterium, and R¹ is n-propyl optionally substituted with deuterium, such as —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃. In one example of this more particular aspect, each of Y¹, Y², and Y³ is deuterium. In another example of this aspect, each of Y¹, Y², and Y³ is hydrogen. In one example of this more particular aspect, each of Y⁴ and Y⁵ is deuterium. In another example of this more particular aspect, each of Y⁴ and Y⁵ is hydrogen.

In one embodiment, R² is phenyl optionally substituted with R³ and optionally substituted with deuterium. In one aspect of this embodiment, R² is phenyl optionally substituted with deuterium and substituted at the 4-position with methyl optionally substituted with deuterium. In one aspect of this embodiment, R² is phenyl substituted with —CD₃ at the 4-position and optionally substituted with deuterium. In one aspect of this embodiment, R¹ is n-propyl optionally substituted with deuterium, such as —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃. In a more particular aspect of this embodiment, R² is phenyl substituted with —CD₃ at the 4-position and optionally substituted with deuterium, and R¹ is n-propyl optionally substituted with deuterium, such as —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃. In one example of this aspect, each of Y¹, Y², and Y³ is deuterium. In another example of this aspect, each of Y¹, Y², and Y³ is hydrogen. In one example of this aspect, each of Y⁴ and Y⁵ is deuterium. In another example of this aspect, each of Y⁴ and Y⁵ is hydrogen.

In one embodiment, each of Y¹, Y², and Y³ is deuterium. In another embodiment, each of Y¹, Y², and Y³ is hydrogen.

In one embodiment, each of Y⁴ and Y⁵ is deuterium. In one aspect of this embodiment, each of Y¹, Y², and Y³ is deuterium. In another aspect, each of Y¹, Y², and Y³ is hydrogen.

In one embodiment, each of Y⁴ and Y⁵ is hydrogen. In one aspect of this embodiment, each of Y¹, Y², and Y³ is hydrogen. In another aspect, each of Y¹, Y², and Y³ is deuterium.

In one embodiment of this invention the compound of Formula I is a compound of Formula Ib:

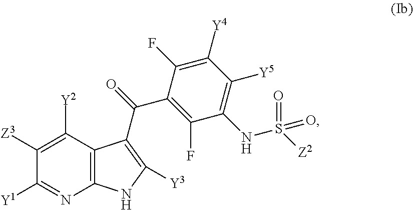

or a pharmaceutically acceptable salt thereof, wherein Z² is n-propyl optionally substituted with deuterium and Z³ is phenyl substituted with chlorine, wherein any hydrogen of Z³ is optionally replaced with deuterium,
with the proviso that at least one of Z² and Z³ comprises deuterium.

In one aspect of this embodiment, Z³ is selected from

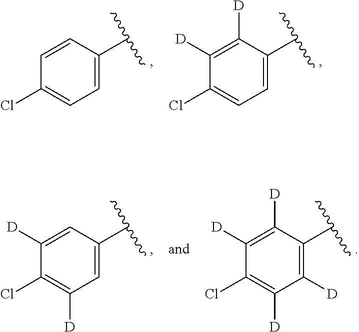

For example, Z³ may be selected from

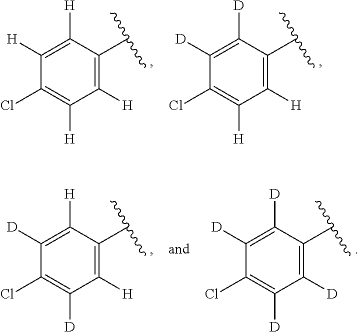

In one embodiment of this invention the compound of Formula I is a compound of Formula Ic:

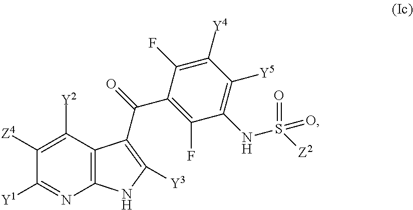

or a pharmaceutically acceptable salt thereof, wherein Z² is n-propyl optionally substituted with deuterium and Z⁴ is phenyl substituted with methyl, wherein any hydrogen of the phenyl or methyl portion of Z⁴ is optionally replaced with deuterium,
with the proviso that at least one of Z² and Z⁴ comprises deuterium.

In one aspect of this embodiment, Z⁴ is selected from

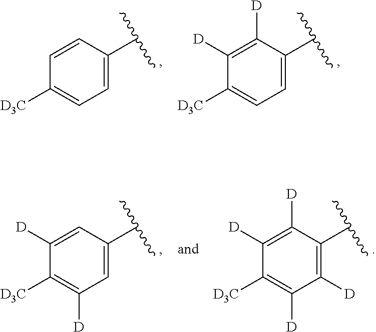

For example, Z⁴ may be selected from

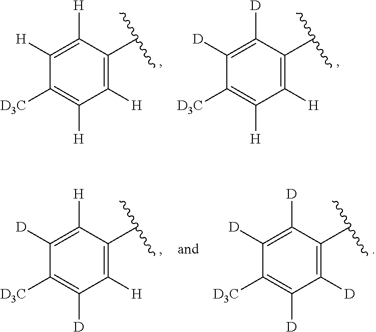

In another set of embodiments, any atom not designated as deuterium in any of the embodiments set forth above is present at its natural isotopic abundance.

Specific examples of a compound of Formula Ib include a compound selected from the group consisting of the following, wherein in each compound below “Cl-Ph” means

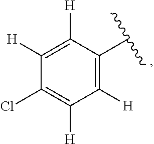

and
(a) in 100a, 101a, 102a, 103a, 104a, 105a and 106a, each of Y¹, Y², Y³, Y⁴, and Y⁵ is deuterium; and
(b) in 100b, 101b, 102b, 103b, 104b, 105b and 106b, each of Y¹, Y², Y³, Y⁴, and Y⁵ is hydrogen:

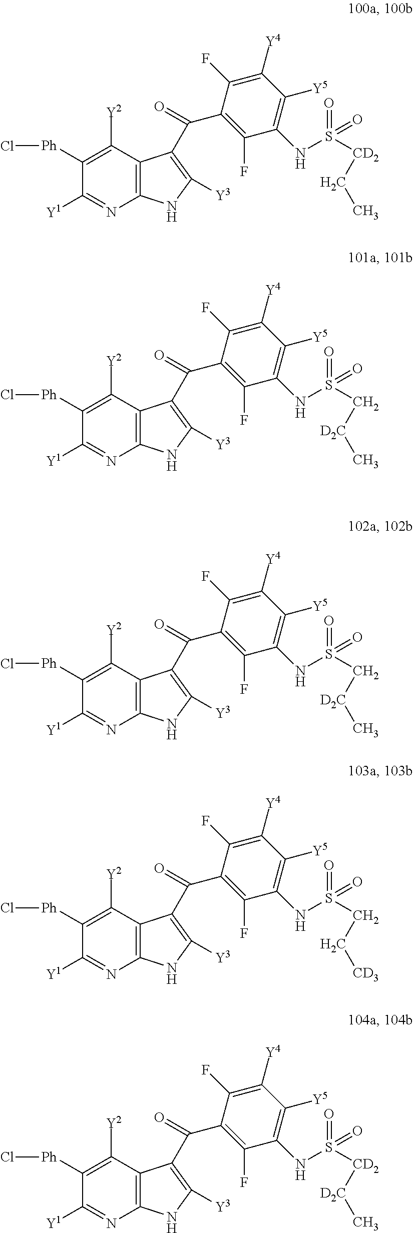

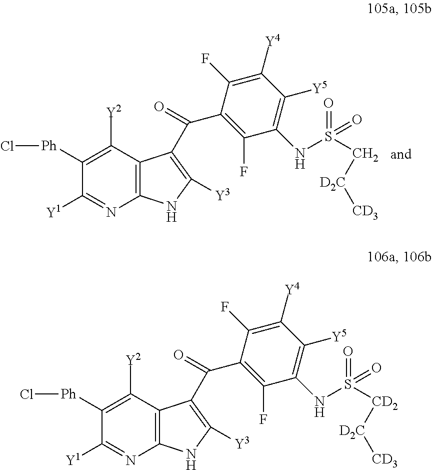

or a pharmaceutically acceptable salt of any of the foregoing.

The synthesis of compounds of Formula I, Ib and Ic may be readily achieved by synthetic chemists of ordinary skill by reference to the Exemplary Synthesis disclosed herein. Relevant procedures analogous to those of use for the preparation of compounds of Formula I, Ib and Ic and intermediates thereof are disclosed, for instance in patent publication WO 2007002325.

### Exemplary Synthesis

Compounds of Formula I, Ib and Ic may be prepared according to the schemes shown below.

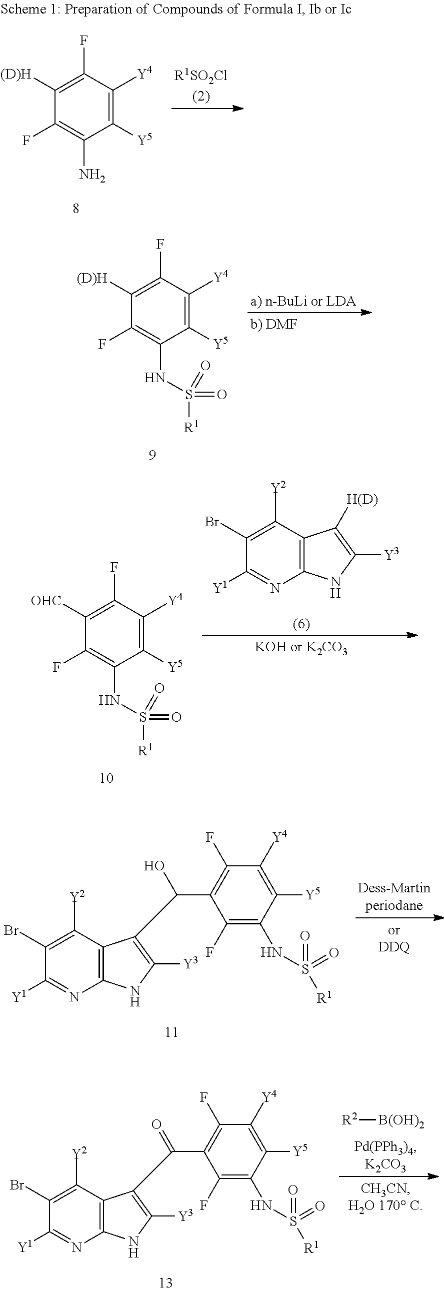

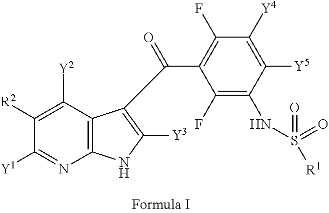

Scheme 1 depicts an example of a route to preparing compounds of Formula I, Ib and Ic. The route is useful, for example, for compounds where R¹ is —CD₂CH₂CH₃ or —CD(CH₃)₂ and R² is -4-chloro-phenyl optionally substituted with deuterium or -4-methyl-phenyl optionally substituted with deuterium. Aniline 8a, wherein Y⁴ and Y⁵ are each H, is commercially available. Aniline 8b, wherein Y⁴ and Y⁵ are each D may be prepared from commercially available perdeutero-1,3-benzenediamine in a manner analogous to that described by Wang et al., Nongyao, 2005, 44(1): 13-15. Appropriately deuterated aniline 8 is treated with appropriately deuterated sulfonyl chloride 2 (see below) to provide sulfonamide 9. Deprotonation with n-butyllithium or LDA followed by reaction with dimethylformamide gives aldehyde 10 (in a manner analogous to what is described in WO 2009012283 and WO 2008079906). Treatment of 10 with potassium hydroxide or potassium carbonate and 6, which can be, for example, commercially available 6(i), gives appropriately deuterated 11. Similarly, treatment with commercially-available potassium deuteroxide and 6, which can be, for example, 6(ii) (prepared from commercially available perdeutero-7-azaindole in a manner analogous to that described by Wu, P. W. et al., JACS, 2006, 128(45): 14426-27), gives appropriately deuterated 11. 6(i) and 6(ii) are shown below:

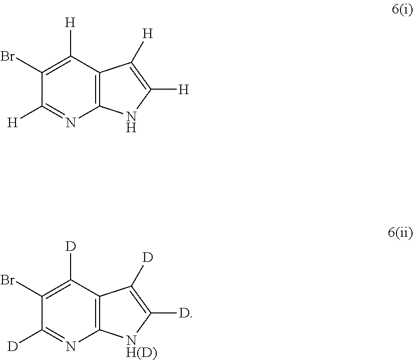

Oxidation of 11 with either Dess-Martin periodinane (WO 2008079906) or DDQ (Tsai, J. et al. Proc. Natl. Acad. Sci. USA 2008, 105(8): 3041-3046) provides 13. Reaction of 13 with (4-chlorophenyl)boronic acid (prepared as described, for example, in Fukuda et al., Tetrahedron (2008), 64(2), 328-338) under Suzuki coupling conditions provides compounds of Formula I and Ib wherein R² is 4-chlorophenyl. Similarly, Reaction of 13 with (4-chloro-tetradeuterophenyl)boronic acid under similar conditions provides compounds of Formula I and Ib wherein R² is 4-chlorotetradeuterophenyl. Reaction of 13 with (4-methylphenyl)boronic acid under Suzuki coupling conditions provides compounds of Formula I and Ic wherein R² is 4-methylphenyl. Similarly, Reaction of 13 with (4-trideuteromethylphenyl)boronic acid under similar conditions provides compounds of Formula I and Ic wherein R² is 4-trideuteromethylphenyl. (4-Chloro-tetradeuterophenyl)boronic acid may be prepared from (4-chloro)tetradeuterobromobenzene analogously to the procedure of Fukuda et al., supra, as illustrated in Scheme 2a. In turn, (4-chloro)tetradeuterobromobenzene may be prepared, for example, as described in Enache, L. A., et al., Bioorganic & Medicinal Chemistry Letters (2009), 19(22), 6275-6279.

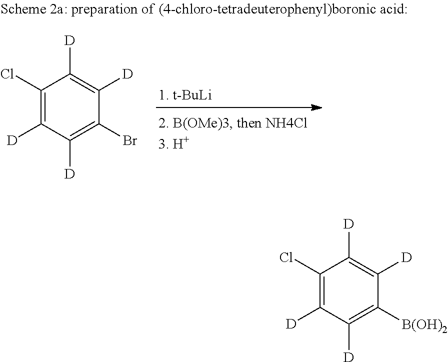

(4-trideuteromethylphenyl)boronic acid may be prepared from (4-trideuteromethyl)bromobenzene, commercially available from Oakwood Products, Inc., analogously to the procedure of Percec, V. et al., J. Am. Chem. Soc. 2007, 129, p. 11265, as illustrated in Scheme 2b:

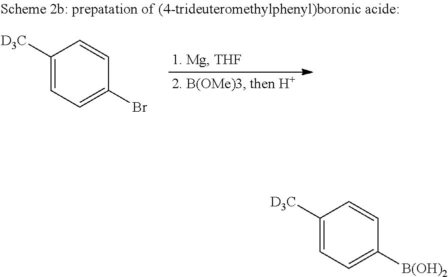

It would be readily apparent to one of skill in the art that compounds of Formula I, Ib and Ic may be generally prepared as described in Scheme 1.

Intermediate 2 may be one of 2a-2g:

Intermediates 2a-2g
CD₃CD₂CD₂SO₂Cl 2a
CD₃CD₂CH₂SO₂Cl 2b
CD₃CH₂CD₂SO₂Cl 2c
CH₃CD₂CD₂SO₂Cl 2d
CD₃CH₂CH₂SO₂Cl 2e
CH₃CD₂CH₂SO₂Cl 2f
CH₃CH₂CD₂SO₂Cl 2g

Intermediates 2a-2g may be prepared from the appropriately deuterated n-propyl thiol in a manner analogous to that described by Prakash, G K S et al., JOC, 2007, 72(15): 5847-50. Starting material for the preparation of 2a, CD₃CD₂CD₂SH, is commercially available. Other appropriately deuterated n-propyl thiols may be prepared from commercially available deuterated n-propyl bromides in a manner analogous to that described by Cossar, B C, et al., JOC, 1962, 27:93-95. Examples of commercially available deuterated n-propyl bromides include CH₃CD₂CD₂Br, CD₃CH₂CD₂Br, CD₃CD₂CH₂Br, CD₃CH₂CH₂Br, CH₃CD₂CH₂Br, and CH₃CH₂CD₂Br. Additional intermediates 2 may be prepared as shown in Scheme 3 below.

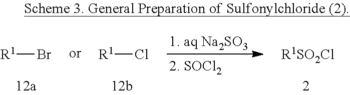

Appropriately deuterated intermediates 2 may be prepared as depicted in Scheme 3 in a manner analogous to that described in patent publication WO 2007140317A2. Conversion of appropriately deuterated alkyl halide 12a or 12b to the corresponding sulfonic acid in the presence of aqueous sodium sulfite followed by treatment with thionyl chloride results in the formation of sulfonylchloride 2. Commercially available alkyl bromides 12a or chlorides 12b, of use in Scheme 3 above, are shown below.

| | | | |
|---|---|---|---|
| CD₃CD₂CD₂Br(Cl) | CH₃CH₂CD₂Br(Cl) | CH₃CD₂CD₂Br | CD₃CH₂CD₂Br |
| CD₃CH₂CH₂Br | CD₃CD₂CH₂Br | CH₃CD₂CH₂Br | |
| (CD₃)₂CDBr(Cl) | CD₃(CH₃)CHBr(Cl) | (CH₃)₂CDBr(Cl) | (CD₃)₂CHBr(Cl) |

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art. Combinations of substituents and variables envisioned by this invention are only those that result in the formation of stable compounds.

### Compositions

The invention also provides pyrogen-free compositions comprising a compound of Formula I, Ib or Ic (e.g., including any of the formulae herein), or a pharmaceutically acceptable salt of said compound; and an acceptable carrier. In one embodiment, the composition comprises an effective amount of the compound of Formula I, Ib or Ic. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

In another embodiment, a composition of this invention further comprises a second therapeutic agent. The second therapeutic agent may be selected from any compound or therapeutic agent known to have or that demonstrates advantageous properties when administered with a compound that selectively inhibits oncogenic B-Raf^{V600E} protein kinase.

Preferably, the second therapeutic agent is an agent useful in the treatment or prevention of a disease or condition selected from neoplastic diseases and associated complications, including chemotherapy-induced hypoxia, gastrointestinal stromal tumors (GISTs), prostate tumors, mast cell tumors (including canine mast cell tumors), acute myeloid leukemia, acute lymphocytic leukemia, chronic myeloid leukemia, multiple myeloma, melanoma, mastocytosis, gliomas, glioblastoma, astrocytoma, neuroblastoma, sarcomas (e.g. sarcomas of neuroectodermal origin), carcinomas (e.g. lung, breast, pancreatic, renal, female genital tract, carcinoma in situ), lymphoma (e.g. histiocytic lymphoma), neurofibromatosis (including Schwann cell neoplasia), myelodysplastic syndrome, leukemia, tumor angiogenesis, and cancers of the thyroid, liver, bone, skin, brain, pancreas, lung (e.g. small cell lung cancer), breast, colon, prostate, testes and ovary. As an example, the disease or condition is selected from melanoma, thyroid cancer, ovarian cancer, and colorectal cancer.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In one embodiment of the pharmaceutical compositions of the invention, the compound of the present invention is present in an effective amount. As used herein, the term “effective amount” refers to an amount which, when administered in a proper dosing regimen, is sufficient to treat a disease or disorder, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 0.01 to about 5000 mg per treatment. In more specific embodiments the range is from about 0.1 to 2500 mg, or from 0.2 to 1000 mg, or most specifically from about 1 to 500 mg. Treatment typically is administered one to three times daily.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

### Methods of Treatment

According to another embodiment, the invention provides a method of treating a patient suffering from, or susceptible to, a disease that is beneficially treated by an inhibitor of oncogenic B-Raf^{V600E} protein kinase comprising the step of administering to said patient an effective amount of a compound of this invention or a pharmaceutically acceptable salt of said compound or a composition of this invention. Such diseases include diseases that are treated by selectively inhibiting oncogenic B-Raf^{V600E} protein kinase.

In another embodiment, any of the above methods of treatment comprises the further step of co-administering to the patient one or more second therapeutic agents. The choice of second therapeutic agent may be made from any second therapeutic agent known to be useful for co-administration with a compound that selectively inhibits oncogenic B-Raf^{V600E} protein kinase. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered (i) together with a compound of this invention as part of a single dosage form (such as a composition of this invention comprising a compound of the invention and an second therapeutic agent as described above) or as separate, multiple dosage forms; or (ii) prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, Ib or Ic, or a pharmaceutically acceptable salt of said compound, alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I, Ib, or Ic or a pharmaceutically acceptable salt thereof for use in the treatment or prevention in a patient of a disease, disorder or symptom thereof delineated herein.

### EXAMPLE

### Example 1

### Synthesis of N-(2,4-difluoro-3-(5-(4-chlorophenyl)-1H-pyrrolo[2,3-b]pyridine-3-carbonyl)phenyl)-1,1,2,2,3,3,3-d₇-propane-1-sulfonamide (106b)

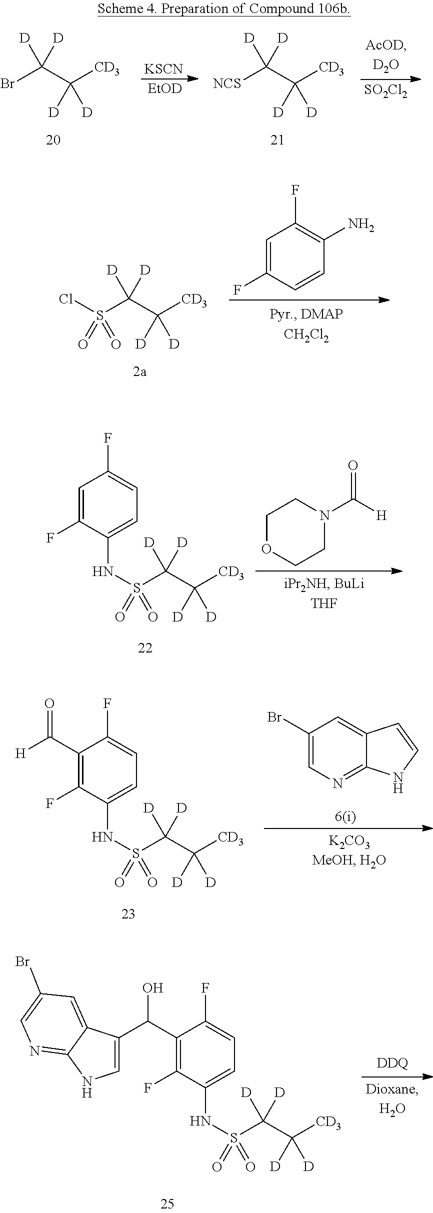

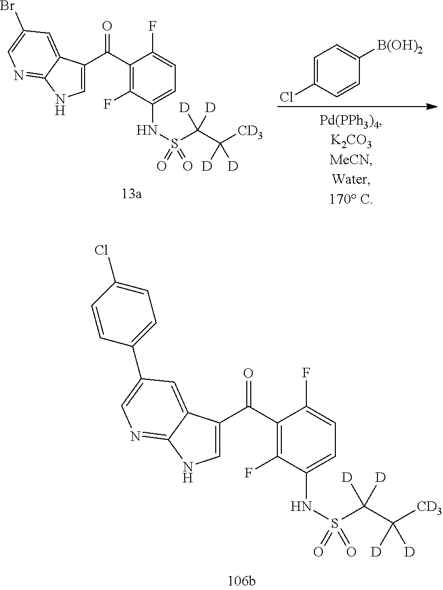

Step 1. 3-Thiocyanato1,1,2,2,3,3,3-d₇-propane (21): To a solution of d₇-bromopropane 20 (0.740 mL, 7.69 mmol, CDN, 98 atom % D) in EtOD (11 mL, Sigma-Aldrich, 99.5+ atom % D) was added potassium thiocyanate (1.12 g, 11.5 mmol). The reaction was heated to reflux and stirred for 3 hours. Upon completion, the reaction was cooled to room temperature, diluted with D₂O (Cambridge Isotope Laboratories, 99.8 atom % D), and extracted with EtOAc (3×50 mL). The organic layers were combined, washed with D₂O, dried (Na₂SO₄), filtered and concentrated under reduced pressure to afford thiocyanate 21 (824 mg, 99%) which was used in the next step without further purification.

Step 2. 1,1,2,2,3,3,3-d₇-Propane-1-sulfonyl chloride (2a): A solution of 21 (824 mg, 7.61 mmol) in AcOD (2.20 mL, 38.1 mmol, Sigma-Aldrich, 99 atom % D) and D₂O (0.230 mL, 11.4 mmol, Cambridge Isotope Laboratories, 99.8 atom % D) was stirred at 50° C. for 30 minutes. Sulfuryl chloride (6.17 mL, 76.1 mmol) was then added dropwise and the reaction continued to stir for an additional 30 minutes. Upon cooling to room temperature the reaction was quenched by the dropwise addition of water (10 mL) and the resulting solution was extracted with EtOAc (3×50 mL). The organic layers were combined, washed with water, dried (Na₂SO₄), filtered and concentrated under reduced pressure to afford 2a (1.14 g, 99%) as a clear oil which was used in the next step without further purification. MS (ESI)) sulfonic acid ion observed: 130.2 [(M−H)⁻].

Step 3. N-(2,4-Difluorophenyl)-1,1,2,2,3,3,3-d₇-propane-1-sulfonamide (22): Sulfonyl chloride 2a (1.14 g, 7.61 mmol) was added dropwise to a solution of 2,4-difluoroaniline (0.740 mL, 7.35 mmol), pyridine (0.615 mL, 7.61 mmol) and DMAP (45.0 mg, 0.370 mmol) in dichloromethane (10 mL) at room temperature. The reaction was stirred at 45° C. for 15 hours then cooled to room temperature, diluted with water and extracted with EtOAc (3×50 mL). The organic layers were combined, washed with brine, dried (Na₂SO₄), filtered and concentrated under reduced pressure. The resulting brown solid was recrystallized with EtOAc/Heptanes to afford sulfonamide 22 (604 mg, 34%) as a tan solid. MS (ESI) 241.1 [(M−H)⁻].

Step 4. N-(2,4-Difluoro-3-formylphenyl)-1,1,2,2,3,3,3-d₇-propane-1-sulfonamide (23): To a solution of N,N-diisopropylamine (0.775 mL, 5.48 mmol) in THF (10 mL) at −78° C. was added dropwise n-butyllithium (2.95 mL, 5.48 mmol, 1.86M solution in hexane). The resulting solution stirred at 0° C. for 1 hour then was cooled to −78° C. and a solution of 7 (604 mg, 2.49 mmol) in THF (5 mL) was added dropwise. After stirring at −78° C. for 4 hours, N-formylmorpholine (0.302 mL, 2.99 mmol) was added and the reaction stirred at room temperature for 15 hours and subsequently quenched with 1N HCl and extracted with EtOAc (3×50 mL). The organic layers were combined, washed with water, dried (Na₂SO₄), filtered and concentrated under reduced pressure. The resulting brown solid was recrystallized from EtOAc/heptanes to afford formylsulfonamide 23 (265 mg, 40%) as a tan solid. MS (ESI) 269.1 [(M−H)⁻].

Step 5. N-(3-((5-Bromo-1H-pyrrolo[2,3-b]pyridin-3-yl)(hydroxy)-methyl)-2,4-difluorophenyl)-1,1,2,2,3,3,3-d₇-propane-1-sulfonamide (25): To a solution of formylsulfonamide 23 (50.0 mg, 0.185 mmol) in MeOH/Water 1:1 (1 mL) was added 5-bromoazaindole 6(i) (30.0 mg, 0.196 mmol, Adesis, Inc.) followed by K₂CO₃ (171 mg, 1.24 mmol). The resulting solution stirred at room temperature for 3 days then was neutralized to pH 7 with 4N HCl and extracted with EtOAc (3×50 mL). The organic layers were combined, washed with water, dried (Na₂SO₄), filtered and concentrated under reduced pressure to afford azaindole 25 (142 mg, 82%) as a tan solid which was used without further purification. MS (ESI) 465.0 [(M−H)⁻].

Step 6. N-(3-(5-Bromo-1H-pyrrolo[2,3-b]pyridine-3-carbonyl)-2,4-difluorophenyl)-1,1,2,2,3,3,3-d₇-propane-1-sulfonamide (13a): 2,3-Dichloro-5,6-dicyano-1,4-benzoquinone (DDQ) (42.0 mg, 0.184 mmol) was added to a solution of azaindole 25 (60.0 mg, 0.142 mmol) in 1,4-dioxane (1.00 mL) and water (0.100 mL). The resulting solution stirred at room temperature for 2 hours then was quenched with sat. NaHCO₃ and extracted with 1:1 THF/EtOAc (3×15 mL). The organic layers were combined, diluted with heptanes (10 mL), dried (Na₂SO₄), filtered through a pad of silica gel and concentrated under reduced pressure to afford ketone 13a. MS (ESI) 463.0 [(M−H)⁻].

Step 7. N-(2,4-Difluoro-3-(5-(4-chlorophenyl)-1H-pyrrolo[2,3-b]pyridine-3-carbonyl)phenyl)-1,1,2,2,3,3,3-d₇-propane-1-sulfonamide (106b): To a solution of N-(3-(5-bromo-1H-pyrrolo[2,3-b]pyridine-3-carbonyl)-2,4-difluorophenyl)-1,1,2,2,3,3,3-d₇-propane-1-sulfonamide (13a; 69.0 mg, 0.148 mmol) in water (0.600 mL) and acetonitrile (1.20 mL) was added 4-chlorophenyl boronic acid (116 mg, 0.740 mmol), K₂CO₃ (122 mg, 0.888 mmol) and tetrakis(triphenylphoshine)palladium(0) (2.00 mg, 1.5×10⁻³ mmol). The resulting solution stirred in a sealed pressure flask at 170° C. for 15 hours was then cooled to room temperature, diluted with water and extracted with EtOAc (3×5 mL). The organic layers were combined, dried (Na₂SO₄), filtered through a pad of silica gel and concentrated under reduced pressure to afford ketone 106b. MS (ESI) 497.0 [(M+H)⁺].

### Example 2

### Evaluation of Metabolic Stability in Human Liver Microsomes

Human liver microsomes (20 mg/mL) are available from Xenotech, LLC (Lenexa, KS). β-nicotinamide adenine dinucleotide phosphate, reduced form (NADPH), magnesium chloride (MgCl₂), and dimethyl sulfoxide (DMSO) are available from Sigma-Aldrich.

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for PLX4032 and the positive control, 7-ethoxycoumarin (1 μM). Testing is done in triplicate.

The in vitro t_{1/2}s for test compounds are calculated from the slopes of the linear regression of % parent remaining (ln) vs incubation time relationship:

in vitro t_{1/2}=0.693/k

k=−[slope of linear regression of % parent remaining (ln) vs incubation time].

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

What is claimed is:

1. A compound of Formula I: 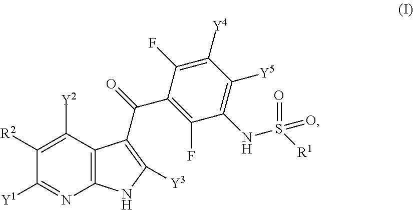
or a pharmaceutically acceptable salt thereof, wherein:
R¹ is C₁-C₄ alkyl optionally substituted with deuterium;
each of Y¹, Y², Y³, Y⁴, and Y⁵ is independently selected from hydrogen and deuterium; R² is C₆-C₁₀ aryl (i) optionally substituted with deuterium and (ii) optionally substituted with halo or with R³;
R³ is C₁-C₄ alkyl optionally substituted with deuterium;
with the proviso that if each of Y¹, Y², Y³, Y⁴, and Y⁵ is hydrogen, then
at least one of R¹, R² and R³ comprises deuterium.

2. The compound of claim 1, wherein R¹ is isopropyl optionally substituted with deuterium.

3. The compound of claim 2, wherein R¹ is —CH(CH₃)₂, —CD(CH₃)₂, —CH(CD₃)₂, or —CD(CD₃)₂.

4. The compound of claim 1, wherein R¹ is n-propyl optionally substituted with deuterium.

5. The compound of claim 4, wherein R¹ is —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃.

6. The compound of any one of claims 1-5, wherein R² is phenyl optionally substituted with halo and optionally substituted with deuterium.

7. The compound of claim 6, wherein R² is -4-chlorophenyl, optionally substituted with deuterium.

8. The compound of any one of claims 1-5, wherein R² is phenyl optionally substituted with R³ and optionally substituted with deuterium.

9. The compound of claim 8, wherein R² is phenyl optionally substituted with deuterium and substituted at the 4-position with methyl optionally substituted with deuterium.

10. The compound of claim 9, wherein R² is -4-trideuteromethylphenyl, wherein the phenyl is optionally substituted with deuterium.

11. The compound of any one of claims 1-10, wherein R¹ is —CH₂CH₂CH₃, —CD₂CH₂CH₃, —CH₂CD₂CH₃, —CH₂CH₂CD₃, —CD₂CD₂CH₃, —CD₂CH₂CD₃, —CH₂CD₂CD₃, or —CD₂CD₂CD₃.

12. The compound of any one of claims 1-11, wherein each of Y¹, Y², and Y³ is deuterium.

13. The compound of any one of claims 1-11, wherein each of Y¹, Y², and Y³ is hydrogen.

14. The compound of any one of claims 1-13, wherein each of Y⁴ and Y⁵ is deuterium.

15. The compound of any one of claims 1-13, wherein each of Y⁴ and Y⁵ is hydrogen.

16. The compound of claim 1, wherein the compound is a compound of Formula Ib: 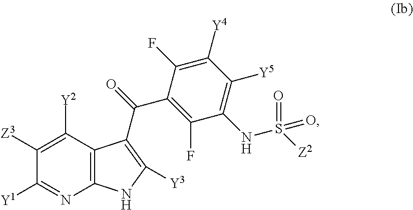
or a pharmaceutically acceptable salt thereof, wherein Z² is n-propyl optionally substituted with deuterium and Z³ is phenyl substituted with chlorine, wherein any hydrogen of Z³ is optionally replaced with deuterium, with the proviso that at least one of Z² and Z³ comprises deuterium.

17. The compound of claim 16, wherein the compound is selected from the group consisting of the following compounds, wherein (a) in 100a, 101a, 102a, 103a, 104a, 105a and 106a, each of Y¹, Y², Y³, Y⁴, and Y⁵ is deuterium; and (b) in 100b, 101b, 102b, 103b, 104b, 105b and 106b, each of Y¹, Y², Y³, Y⁴, and Y⁵ is hydrogen: 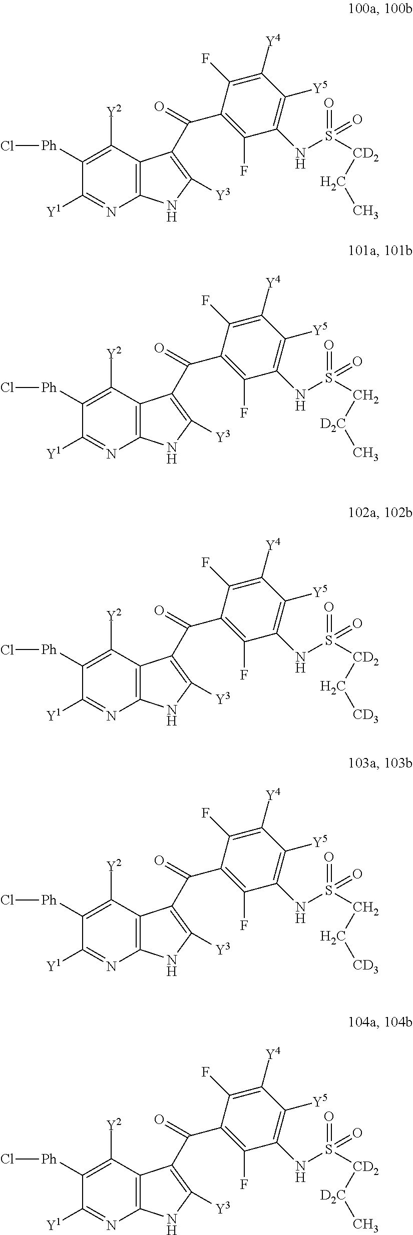 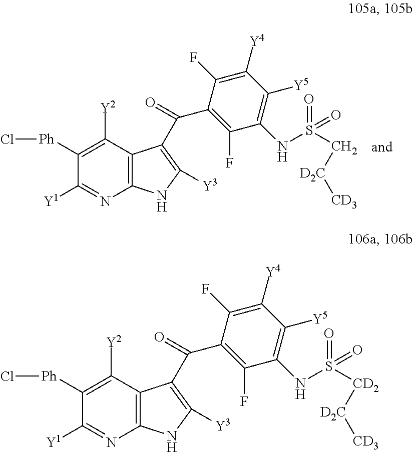
or a pharmaceutically acceptable salt of any of the foregoing.

18. The compound of claim 1, wherein the compound is a compound of Formula Ic: 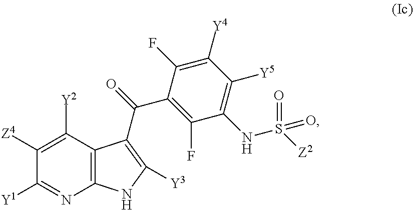
or a pharmaceutically acceptable salt thereof, wherein Z² is n-propyl optionally substituted with deuterium and Z⁴ is phenyl substituted with methyl, wherein any hydrogen of the phenyl or methyl portion of Z⁴ is optionally replaced with deuterium, with the proviso that at least one of Z² and Z⁴ comprises deuterium.

19. The compound of any one of claims 1 to 18, wherein any atom not designated as deuterium is present at its natural isotopic abundance

20. A pyrogen-free pharmaceutical composition comprising a compound of any one of claims 1 to 19 or a pharmaceutically acceptable salt of said compound; and a pharmaceutically acceptable carrier.

21. The composition of claim 20 additionally comprising a second therapeutic agent useful in the treatment or prevention of a disease or condition selected from melanoma, thyroid cancer, ovarian cancer, and colorectal cancer.

22. A method of treating a patient suffering from, or susceptible to, a disease or condition selected from chemotherapy-induced hypoxia, gastrointestinal stromal tumors (GISTs), prostate tumors, mast cell tumors, acute myeloid leukemia, acute lymphocytic leukemia, chronic myeloid leukemia, multiple myeloma, melanoma, mastocytosis, gliomas, glioblastoma, astrocytoma, neuroblastoma, sarcomas, carcinomas, lymphoma, neurofibromatosis, myelodysplastic syndrome, leukemia, tumor angiogenesis, and cancers of the thyroid, liver, bone, skin, brain, pancreas, lung, breast, colon, prostate, testes and ovary, comprising the step of administering to the patient in need thereof an effective amount of a composition of claim 20.

23. The method of claim 22, wherein the disease or condition is selected from melanoma, thyroid cancer, ovarian cancer, and colorectal cancer.
